(19) United States
(12) Patent Application Publication
(10) Pub. No.: US 2007/0079450 A1
(43) Pub. Date: Apr. 12, 2007

(54) Products and processes for removing makeup from the hair after a ligand-receptor treatment

(76) Inventors: Gabin Vic, Venette (FR); Aude Livoreil, Paris (FR); Bruno A. Bernard, Neuilly-Sur-Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/556,812

(22) PCT Filed: May 6, 2004
(86) PCT No.: PCT/FR04/01090

§ 371 Date: Nov. 21, 2006

(30) Foreign Application Priority Data
May 14, 2003 (FR) 03/05775

(51) Int. Cl. A61K 8/00; A61K 8/18

(52) U.S. Cl. 8/405; 424/70.1

(57) ABSTRACT

The invention concerns methods for removing make-up from keratinous materials which consist in applying, after treating the fiber with a ligand-receptor system, at least one ligand and/or at least one salt. The invention also concerns make-up compositions for keratinous materials comprising, at least one ligand and at least one compound selected among salts and amino acids.

The present invention relates to processes for removing makeup from keratin materials, which consists in applying, after treating the fiber with a ligand-receptor system, at least one ligand and/or at least one salt.

This invention also relates to compositions for removing makeup from keratin materials comprising at least one ligand and at least one compound chosen from salts and amino acids.

Various cosmetic technologies have already been described for depositing cosmetic active agents onto the surface of the hair. These deposits use, for example, the strong forces of association that exist between a ligand and its biological receptor. This ligand-receptor association is spontaneous, specific and long-lasting.

Patent EP 0 634 161 especially claims the use of anti-keratin antibodies for coloring the hair. Patent EP 0 570 583 claims the use of anti-keratin antibodies for treating damaged hair. The Applicant has also claimed in patent applications FR 01/17074, FR 02/04952 and FR 02/11782 the use of exogenous hair antigens in haircare applications. These treatments allow long-lasting benefits to be provided under mild conditions that do not degrade the hair fiber.

However, these deposits of material are difficult to remove on shampooing and no process exists for rapidly restoring, without degradation,. the initial state of the fiber before deposition. Regaining this initial state is, in point of fact, particularly important in order to be able to reapply cosmetic treatments such as dyeing or permanent-waving to the fiber while avoiding any interference with the ligand-receptor deposit deposited on the fiber beforehand, but also to be able optionally to give the hair a further makeup treatment with a new deposit based on ligand-receptor.

The Applicant has discovered, surprisingly, that if keratin materials that have been subjected to a deposition of material by the ligand-receptor technology are treated with a product comprising at least one organic or mineral salt, or at least one ligand, the initial appearance and state of the hair fiber before the deposition of material can be restored.

A subject of the invention is also compositions for removing makeup from the hair, comprising at least one ligand and at least one compound chosen from salts (organic or mineral) and amino acids.

Other subjects of the present patent application will emerge on reading the description and the examples that follow.

The compositions containing the salts or ligands according to the invention may also contain a surfactant.

The compositions according to the invention may also contain a soluble polymer.

The cosmetically acceptable medium may consist solely of water, or of a solvent or mixture of solvents, or of a mixture of water and of one or more cosmetically acceptable solvents such as a C₁-C₄ lower alcohol, for instance ethanol, isopropanol, tert-butanol or n-butanol; alkylene glycols, for instance propylene glycol, or glycol ethers.

The pH of these compositions according to the invention is between 1 and 11 and preferably between 2 and 7. These compositions preferably contain between 0.1% and 90% and preferably between 1% and 20% of salts. When one or more ligands are present, their concentration is preferably between 0.01% and 40% and preferably between 0.1% and 10% of the total weight of the composition.

These compositions according to the invention may also contain additives such as natural or synthetic, anionic, amphoteric, zwitterionic, nonionic or cationic, associative or nonassociative polymeric thickeners, nonpolymeric thickeners, for instance acids or electrolytes, anionic, nonionic or amphoteric surfactants, nacreous agents, opacifiers, fragrances, dyes, mineral or organic particles, preserving agents and pH stabilizers.

A person skilled in the art will take care to select the optional additives and the amount thereof such that they do not harm the properties of the components of the product.

These additives are present in the product according to the invention in an amount ranging from 0 to 20% by weight relative to the total weight of the product.

The products according to the invention may be in the form of more or less thick solutions, creams, fluid gels or mousses. They may be packaged in aerosol form.

A first subject of the invention is thus a set of processes for removing makeup from keratin materials and in particular the hair.

A first process according to the invention consists in applying, after treating the hair with a ligand-receptor system, a ligand or a salt and then in rinsing after an optional leave-in time.

A second process according to the invention consists in applying, after treating the hair with a ligand-receptor system, at room temperature or at elevated temperature, an effective amount of a mixture of at least one ligand and of at least one salt and then in rinsing after a certain leave-in time.

It is possible to first apply a salt solution and then a ligand solution. In one variant of the last process, the products may be left on the hair. The hair may optionally be washed with water or with a shampoo or dried between the two applications, or only at the end.

In a second variant of the last process according to the invention, it is possible to first apply a ligand solution and then a salt solution. The products may be left on the hair. The hair may optionally be washed with water or with a shampoo or dried between the two applications or only at the end.

The compositions of this product may be applied at elevated temperature, ie at a temperature above 40° C.

These processes make it possible, on hair that has been subjected to a deposition of material via the ligand-receptor technology, to dissociate the ligand and the receptor, to remove the deposit on the hair and thus to recover the initial state of the hair fiber.

The makeup-removing processes according to the invention are applicable to any biological ligand/receptor couple, for instance:

biotin/avidin, biotin/streptavidin and biotin/neutravidin couples

antigen-antibody couples, the antibodies possibly being fragments of the Fab, Fv or scFv type, etc.,

carbohydrate-lectin couples (Cellulose Binding Domain CBD, concanavalin, etc.),

substrate-enzyme couples (glutathione S-transferase and glutathione, etc.),

polynucleotide couples (recognition of DNA strands, recognition of RNA strands),

couples of proteins modified with a (His)₆ group and certain divalent cations (Cu, Ni or Zn),

polysaccharide couples (cellulose and guar gum, etc.).

These biological ligand/receptor couples are described in patent applications EP 634 161 and EP 570 583 and in the patent applications corresponding to the filings FR 0 117 074, FR 0 204 952 and FR 0 211 782.

Preferably, any ligand capable of complexing with the receptor present on the fiber to be cleansed of makeup, with a dissociation constant of less than or equal to 0.1 at 25° C., will be used as makeup-removing ligand.

One especially advantageous process consists in combining the treatment according to the invention with existing fiber treatments such as permanent reshaping of the hair, oxidation dyeing, bleaching, shampooing or the application of a styling composition.

The ingredients required for the invention (salts or ligands) may be introduced within hair treatment formulations.

Another process according to the invention consists in removing makeup from keratin materials according to one of the processes described above and in following this step with a second step directed towards making up hair again according to the processes mentioned above.

For the purposes of the present invention, the term “salt” means any compound of ionic structure resulting from the action of an acid on a base or of an acid or a base on a metal. The salts that may be used according to the invention may be sodium, potassium, calcium, magnesium, ammonium, lithium, cesium, rubidium or barium and in the form of chloride, sulfate, phosphate, sulfonate, iodide, bromide, cyanate or perchlorate. The salts of organic and mineral acids may also be used, for instance sodium acetate, sodium trichloroacetate or sodium perchlorate, and also the salts of physiologically acceptable organic and mineral bases, for instance monoethanolamine citrate or ammonium lactate, guanidine chloride or glycine chloride, and urea chloride.

For the purposes of the present invention, the term “ligand” means any compound capable of complexing with a second compound known as a receptor, with a dissociation constant of less than or equal to 0.1 at 25° C.

Preferably, a salt whose solubility in water at 25° C. and at atmospheric pressure is greater than or equal to 1% will be used.

The ligands according to the invention comprise, for example, biotin, iminobiotin, soluble keratin, polynucleotides, antigens, sugars and derivatives thereof.

A second subject of the invention is a composition containing at least one ligand and at least one compound chosen from salts (organic or mineral) and amino acids.

The ligands and salts are the same as those described above. Their working concentrations are also the same.

The amino acids that may-be used in these compositions may be of natural or synthetic origin. The acid function may be a carboxylic acid function or a sulfonic acid function. The amino acids according to the invention may comprise one or more identical or different acid functions and one or more amino functions.

In a nonlimiting manner, as amino acids that may be used according to the invention, mention may be made of glycine, histidine, lysine, arginine, ornithine, proline, aspartic acid and taurine.

When one or more amino acids are present, their concentration ranges from 0.01% to 20% and preferably between 0.05% and 10% of the total weight of the composition.

The additives that may be present and the galenical forms of these compositions are the same as those described above.

The examples that follow are intended to illustrate the invention without, however, being limiting in nature.

### EXAMPLE 1

### Removal of Makeup from Hair Carrying a Deposit of Latex Beads

Latex beads bearing biotin groups (Sigma L8155) were deposited onto white hairs precoated with avidin. The makeup removal process according to the invention was then used to remove this deposit of beads.

Step 1: Preparation of the Avidin-Coated Hairs

Five white hairs are incubated in 800 μl of the avidin solution (Sigma at 1.5 mg/ml in a pH 7.2 phosphate buffer). After incubation for 24 hours, rinsing is performed in several steps:

1 rinse with 2 ml of a phosphate buffer solution—Tween 0.05%

1 rinse with 2 ml of a phosphate buffer solution

3 rinses with 2 ml of distilled water

Step 2: Placing of the Hair in Contact with the Biotinlyated Latex Beads

The white hairs are incubated in 1 ml of a solution of latex beads in suspension. The solution is obtained by diluting (1/100) a suspension sold by Sigma (L8155) After incubation for 1 hour, rinsing is performed as described above. The deposition of the beads on the surface of the hair is confirmed by scanning electron microscopy.

Step 3: Removal of the Latex Beads

3.1 Use of a Biotin Solution

The white hairs coated with latex beads are incubated in 1 ml of a 500 μg/ml biotin solution in a pH 7.2 phosphate buffer. After incubation for 1 hour, rinsing is performed as described above. Analysis of the surface of the hairs by electron microscopy shows that the concentration of latex beads on the surface has greatly decreased, reflecting “makeup removal” under the action of biotin.

3.2 Use of an NaCl Solution (1M)

The white hairs coated with latex beads are incubated in 1 ml of an NaCl solution (1M) in a pH 7.2 phosphate buffer. After incubation for 1 hour, rinsing is performed as described previously. Analysis of the surface of the hairs by electron microscopy shows that the concentration of latex beads on the surface has greatly decreased, reflecting “makeup removal” under the action of NaCl.

1. A process for removing makeup from keratin materials, which consists in applying, after treating the fiber with a ligand-receptor system, at least one ligand and/or at least one salt and then in rinsing after an optional leave-in time.

2. The process for removing makeup from keratin materials as claimed in claim 1, in which a composition containing a ligand in a cosmetically acceptable medium is applied.

3. The process for removing makeup from keratin materials as claimed in claim 1, in which a composition containing a salt in a cosmetically acceptable medium is applied.

4. The process for removing makeup from keratin materials as claimed in claim 1, characterized in that a composition containing both a ligand and a salt in a cosmetically acceptable medium is applied.

5. The process for removing makeup from keratin materials as claimed in claim 1, which consists in applying a composition containing a ligand in a cosmetically acceptable medium and a composition containing a salt in a cosmetically acceptable medium sequentially either simultaneously or staggered over time to the keratin materials and then in rinsing after an optional leave-in time.

6. The process for removing makeup from keratin materials as claimed in claim 5, in which the keratin materials are washed with water or with a shampoo between the applications of the two compositions.

7. The process for removing makeup from keratin materials as claimed in claim 5 or 6, in which the keratin materials are dried between the applications of the two compositions.

8. The process for removing makeup from keratin materials as claimed in any one of claims 1, 2 and 4 to 7, characterized in that, in the composition containing the ligand, said ligand is capable of complexing with the receptor present on the fiber to be cleansed of makeup, with a dissociation constant of less than or equal to 0.1 at 25° C.

9. The process for removing makeup from keratin materials as claimed in any one of claims 1, 2 and 4 to 8, characterized in that said ligand is a biotin, an iminobiotin, a soluble keratin, a polynucleotide, an antigen or a sugar, or a derivative thereof.

10. The process for removing makeup from keratin materials as claimed in any one of claims 1 to 9, characterized in that it comprises a prior step consisting of a cosmetic treatment chosen from the application of reducing agents or oxidizing agents for the permanent reshaping of the hair, oxidation dyeing, bleaching, shampooing or the application of a styling composition.

11. The process for removing/reapplying makeup from/to keratin materials as claimed in any one of claims 1 to 10, characterized in that it comprises a subsequent step that consists in making up the keratin fibers again by treating the fiber with a ligand-receptor system.

12. A composition for removing makeup from keratin materials, comprising, in a cosmetically acceptable medium, at least one ligand and at least one compound chosen from organic or mineral salts and amino acids.

13. The composition as claimed in claim 12, characterized in that it contains from 0.1% to 90% by weight of salts relative to the total weight of the composition.

14. The composition as claimed in claim 12 or 13, characterized in that it contains from 1% to 20% by weight of salts relative to the total weight of the composition.

15. The composition as claimed in any one of claims 12 to 14, characterized in that the ligand is preferably a biotin, an iminobiotin, a soluble keratin, a polynucleotide, an antigen or a sugar, or a derivative thereof.

16. The composition as claimed in any one of claims 12 to 15, characterized in that it contains from 0.01% to 40% by weight of ligand relative to the total weight of the composition.

17. The composition as claimed in any one of claims 12 to 16, characterized in that it contains from 0.1% to 10% by weight of ligand relative to the total weight of the composition.

18. The composition as claimed in one of claims 12 to 17, characterized in that the amino acids are of natural or synthetic origin.

19. The composition as claimed in one of claims 12 to 18, characterized in that the amino acids are preferably glycine, histidine, lysine, arginine, ornithine, proline, aspartic acid or taurine.

20. The composition as claimed in any one of claims 12 to 19, characterized in that it contains from 0.01% to 20% by weight of amino acids relative to the total weight of the composition.

21. The composition as claimed in any one of claims 12 to 20, characterized in that it contains from 0.05% to 10% by weight of amino acids relative to the total weight of the composition.

22. The composition as claimed in any one of claims 12 to 21, containing a surfactant.

23. The composition as claimed in any one of claims 12 to 22, containing a soluble polymer.

24. The composition as claimed in any one of claims 12 to 23, characterized in that it contains one or more additives.

25. The composition as claimed in any one of claims 12 to 24, characterized in that it contains from 0 to 20% by weight of additives relative to the total weight of the composition.

26. The composition as claimed in any one of claims 12 to 25, characterized in that it is in the form of a more or less thick aqueous, alcoholic or aqueous-alcoholic solution, a cream, a fluid gel or a mousse, or optionally packaged in aerosol form.
